(19) United States
(12) Patent Application Publication
(10) Pub. No.: US 2015/0314085 A1
(43) Pub. Date: Nov. 5, 2015

(54) VAPOR MEDICINE DISPENSING AND NANO-MIST LUNG CLEANSER SYSTEM

(71) Applicant: Isaac Banoun, Los Angeles, CA (US)

(72) Inventor: Isaac Banoun, Los Angeles, CA (US)

(21) Appl. No.: 14/704,506

(22) Filed: May 5, 2015

Related U.S. Application Data
(60) Provisional application No. 61/988,507, filed on May 5, 2014.

(51) Int. Cl. A61M 11/04; A61M 15/06; A61M 16/20; A61M 15/00

(52) U.S. Cl. CPC A61M 11/042; A61M 15/0021; A61M 15/06; A61M 16/208; A61M 2202/04; A61M 2205/02; A61M 2205/3368; A61M 2205/82

(57) ABSTRACT

A system, accompanying apparatuses and methods relating to an oral, nasal or propellant enhanced mist delivery, drug delivery and lung cleansing apparatuses and having for an object to provide a safe and harmless means for and method of delivering medication with heated, moist, flavored air; or by inhaling warm medication like methanol into the lungs in case of a respiratory ailment under direction of a physician. Additionally, all state of the art medicines are envisioned for delivery with the system.

### CROSS REFERENCE TO RELATED APPLICATION

This application takes priority from and claims the benefit of U.S. Provisional Patent Application Ser. No. 61/988,507 filed on May 5, 2015, the contents of which are herein incorporated by reference.

### FIELD OF THE INVENTION

The present system relates to medical systems for delivery of drugs, medications, cleansers, heated, moist, or flavored air.

### BACKGROUND OF THE INVENTION

Drug delivery refers to approaches, formulations, technologies, and systems for transporting a pharmaceutical compound in the body as needed to safely achieve its desired therapeutic effect. It may involve scientific site-targeting within the body, or it might involve facilitating systemic pharmacokinetics; in any case, it is typically concerned with both quantity and duration of drug presence. Drug delivery is often approached via a drug's chemical formulation, but it may also involve medical devices or drug-device combination products. Drug delivery is a concept heavily integrated with dosage form and route of administration, the latter sometimes even being considered part of the definition.

Concurrent drug delivery technologies modify drug release profile, absorption, distribution and elimination for the benefit of improving product efficacy and safety, as well as patient convenience and compliance. Drug release is from: diffusion, degradation, swelling, and affinity-based mechanisms. Most common routes of administration include the preferred non-invasive perioral (through the mouth), topical (skin), trans mucosal (nasal, buccal/sublingual, vaginal, ocular and rectal) and inhalation routes.

Many medications such as peptide and protein, antibody, vaccine and gene based drugs, in general may not be delivered using these routes because they might be susceptible to enzymatic degradation or cannot be absorbed into the systemic circulation efficiently due to molecular size and charge issues to be therapeutically effective. For this reason many protein and peptide drugs have to be delivered by injection or a nano-needle array.

For example, many immunizations are based on the delivery of protein drugs and are often done by injection. Current efforts in the area of drug delivery include the development of targeted delivery in which the drug is only active in the target area of the body (for example, in cancerous tissues) and sustained release formulations in which the drug is released over a period of time in a controlled manner from a formulation. In order to achieve efficient targeted delivery, the designed system must avoid the host's defense mechanisms and circulate to its intended site of action. Types of sustained release formulations include liposomes, drug loaded biodegradable microspheres, and drug polymer conjugates.

### SUMMARY OF THE INVENTION

The instant apparatus, system and accompanying methods, as illustrated herein, is clearly not anticipated, rendered obvious, or even present in any of the prior art mechanisms, either alone or in any combination thereof. Therefore, it is an object of the instant invention to provide various systems and accompanying apparatuses for oral, nasal or propellant mist enhanced delivery and lung cleansing system and associated apparatuses.

The present system, accompanying apparatuses and methods relate to an oral, nasal or propellant enhanced mist delivery and lung cleansing apparatuses and has for an object to provide a safe and harmless means for and method of delivering medication with heated, moist, flavored air; or by inhaling warm medication like methanol into the lungs in case of a respiratory ailment under direction of a physician.

It is also an objective to introduce a system including a primary use of life extension and pain management. Another object of the system is to provide an article of manufacture wherein air may be drawn through a porous substance or filter mechanism of a cartridge which has been moistened with a chemically harmless flavoring preparation.

An additional objective of the instant system is to combine moisture and taste wherein the moist and flavored air passes through a section of the device heated by a suitable heating element in order that warm, moist and flavored air is drawn into the mouth of the user, and if desired, into the lungs of the user.

A further object of the instant system is to provide a nano-vaporization system smokeless for circulating the fluid around the heating element in a turbulent manner to suitably raise the temperature of the inhalant mixture, with the purpose that the temperature of the flavored air.

A further object is to insulate the heat source as the mechanism may be too hot for the user to hold as well as to introduce a nasal adapter device to be utilized with instant vapor medicine delivery system, which can be adapted for both human and animal applications. An additional object is to introduce a nano-misting or nano-mist system, which can be adapted for both human and animal applications.

A further object of the instant system is to introduce an apparatus with the capability of delivering water, menthol, eucalyptus or glycerol beads of a micro size when compared to current vaporizers by Vicks® or other such systems which deliver large water drops that don't go deep in the lungs. Moreover, concurrent inhalers and nebulizers do not deliver droplets measured in nanometers.

An added and prominent objective of the instant device is to introduce a one way valve to prevent transmission of disease and a filtration system to protect against potential contamination. And, an unexpected result of experimentation with drug delivery processes focuses on the positive utilization of the by-product vapor alone to clean, clear and soothe the lungs and enhance lung functions with the clean steam with small particle size.

An unexpected result of nicotine vaporizers is that the vapor can carry beneficial drugs that actually treat diseases as well as administer treatments and therapies. Unlike today's existing technology which is used as a cigarette cessation or replacement product containing the carcinogen nicotine and in no way is it used or meant to be for the benefits, promotion of health in fact curing or pain managements here cited. You are in effect steam cleaning your lungs and thus entails a whole new entry point for these drugs into the body. Also introduced, a plain water vapor, with nothing added, from these devices, was observed to open the lungs and allow the user to breathe better.

The varied the size of vapor droplets to suit the molecule size of drugs using the existing technology heat control can lead to immediate migraine treatment with such drugs Sumitriptan®. It was not expected that existing devices can work for headaches and other pain management, such as NSAIDs for arthritis.

In additional embodiments, the system may be design to utilize pharmaceutical prescribed drugs (other not nicotine) and caffeine may be help reduce the boiling point or volatilization of another drug. It can also be used for energy vapor markets to replace the energy drink space. Unexpected was that these devices deposit drugs under tongue and in all mouth receptors so delivery is not only through lungs. Different than current use which is intended for lung delivery.

Further addressing the nasal vaporizing as another point of entry totally different than concurrent systems, this would be beneficial to treat many things nasally as well as another entry point to the lungs that is not done using current tech. Also, replaces nasal spray.

Intranasal is a good area for low dose potent drugs, since surface area is normally too small. An additional route for vaccines since you need very little to obtain an antigenic immune response. Thus the instant system allows the user to administer gene therapy as well as viruses and vaccines through the lungs.

Additionally, the system may be utilized for diagnostics and may deliver small molecule drugs as deep as this into the lungs. The depth and reach of drug deposition is unique as the stomach is bypassed stomach for efficacy, purity and speed and thus the drugs or medicines are inherently purer. Thus, mixtures or cocktails can be blended. For example, adding caffeine for energy and to lower the vaporizing temp for other drugs that will not survive the heat.

Aromatherapy usage can clearly provide more focused attention and can be part of our nutritional or neutraceuticals. Wine and alcohol mixed with flavors and glycol or other carriers. Any and all compounds and mixtures may be utilized to cleanse lungs, including common menthol. Sublingual, and mouth and lung combination absorption is possible, including B12 absorption. The vaped B12 will be absorbed thru the mouth and the lungs providing extra energy to the body.

Further, nacetyl lcysteine may be vaped into the lungs and combining this with a nonionic surfactant like polysorbate 80 (TWEEN 80) to create the best available compound which will also be mucolytic. Additives for infections may also be utilized. Thus, all nutraceuticals may be employed, again because the process is in liquid form. And a further combination of mucolytic drugs, cysteine and a non-ionic surfactant (TWEEN 80, poysorbate or other non-ionic surfactants can also be used. The system can therefore replace nasal spray because it delivers a micro-aerosolized dose of therapeutic medication.

Thus, the instant system to deliver drugs and other actives via various routes where other delivery systems have failed or require injections. The delivery system allows drugs to be vaporized (vaped) and efficiently delivered to the lungs, through the nasal route and mouth, including sublingual and across other mucosal membranes. The target market includes delivering efficiently nutraceuticals, herbals, prescription and non-prescription drugs including vitamins, vaccines, anti-pyretics, pain (narcotic and non-narcotic including migraine headaches, diabetes, cystic fibrosis, drugs for obesity and anti-ageing, in a vaporized state. Several patents have been filed to include over-the-counter (OTC) drugs, nutraceuticals, prescription drugs such as nitroglycerin, sildenafil (Viagra, and other similar drugs), active ingredients of Vicks VapoRub and recreational and lifestyle style improving drugs. All drugs in a solvent system that can be vaporized and are heat stable under the E-cig temperatures are included. Vaccines can be delivered in developing countries in a cost-effective manner without cross contamination by changing the disposable tip attached to the device.

Additionally, in one embodiment, the instant device will be unique for medicine as it will comprise a one-way fill valve and thus operate as a one way inhaler to prevent spread of disease if device is being shared, and HEPA® and other filters or better. The fill valve will be spring-loaded may be torsion or other such mechanism as well and which allows the cartridge to be filled with a “needle-point” bottle. The cartridge may be a disposable tip with a one way valve.

For the instant system, this will allow for the cartridge to be filled with medication, while preventing the “patient” from extracting the solution from the cartridge. A specific voltage may to be preset on the battery device specific for each medication, along with an automatic shut-off feature preventing the solution from reaching a defined maximum temperature.

In additional embodiments, steroids, testosterone alcohol, cortisone, prednisone may be administered. Further and even blowing it out in a fine mist to cure large areas of the outside of the body. Can administer burn and many therapies or actual drugs like Botox. Furthermore, a vaporizing vaccine is introduced and also the clearance of lungs due to Propylene Glycol, using Camphor in small particle emission and even vaping of antibiotics to treat pneumonia rather quickly, and a process for treating lung disease with herbs.

The instant system can thus transport antitussive herbs to reduce respiratory spasms; expectorant herbs loosen mucus; demulcent herbs sooth irritated tissue; and antimicrobial herbs resolve infections. For transportation of drugs, in the instant system they may not have to be frozen as they are submerged in the VG/PG or other carrier, thus buried and protected. Also process patents for the drugs that have never been processed in this manner. The drugs are processed through the lungs, not IV, Pills, Powder inhalers, or nebulizers. The system also incorporates sublingual administration as the vapor effects all receptors of the mouth.

Moreover, many processes for the treating of disease and introducing drugs are herein illustrated, for example the system may be utilized to assist breathing for asthma patients. Further, in any of the mixture, the addition of a small quantity of water in the mix to, can prevent dehydration a recurring occurrence with VG/propylene glycol non-aqueous systems. The quantity of the water will be adjusted so that we do not get precipitation of the active. If required, additional ingredients will be added such that the system delivers with a clear mixture or a colored mixture as required for medical or diagnosis purposes. Thus, the instant system represents discovered a new way to improve the current VG/PG mix and transport of all known drugs using an enveloping solution like PG/VG.

Further, Lavender oil, which possesses a scent with a calming effect which may aid in relaxation and the reduction of anxiety and stress may be employed. Lasea capsules containing Lavender oil with a high amount of linalool and linayl acetate termed as Silexan by the manufacturer are approved as an anxiolytic in Germany. The approval is based on the finding that the capsules are comparable in effect to low-dose lorazepam. Additionally, all of the systems herein are adaptable to veterinary use.

Moreover, the instant system may cover all parenteral and non-parenteral routes for administering drugs. The parenteral routes include intravenous, intramuscular, subcutaneous and intradermal. The non-parenteral routes include oral, sublingual, buccal, nasal and vaginal. Drugs such as Bayer®, baby aspirin, and other drugs for migraine and headache, as well as, Pfizer® Viagra® when delivered sublingually map provide biological stability and rapid absorption of the drug.

Yet another object of the present system is to provide a lung cleansing treatment that cleans and clears lungs due to its depth of reach into the smallest airways. Lung cleansing treatment works because the size of the droplet created by the current system is measured in nanometers, and droplets at this size can go to the smallest of airways in the lungs, where menthol and other ingredients inside these particles can be delivered. The droplets are able to attach themselves to the cold receptors in the lining of the airways, which creates shrinkage. The skin retracts as the menthol opens the airways, creating greater breathing capacity. The smallest size of the droplets is key for reaching areas in hard to reach places within the lungs. Most inhaled corticoid drug particles, due to their large size, only make it to half of the lungs, and most of the powder stays in the throat. The nano-particles created by the current system are thus so small in stature as to penetrate the alveoli, and thus proceed where powdered drugs are unable.

The current system generates a mist that as stated is composed of tiny droplets in the low nanometer range (menthol in a gaseous state). The menthol is soothing and cooling in a gaseous state that on inhalation reaches the deep innermost portions of the lungs.

There has thus been outlined, rather broadly, the more important features of the versatile systems, apparatuses and accompany methods in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carries out in various ways. Also, it is to be understood that the phraseology and terminology employed herein are for the purpose of description and should not be regarded as limiting.

These, together with other objects of the invention, along with the various features of novelty, which characterize the invention, are pointed out with particularity in the claims annexed to and forming a part of this disclosure. For a better understanding of the interactive systems, apparatuses and accompany methods, the operating advantages and the specific objects attained by usage, reference should be made to the accompanying drawings and descriptive matter in which there are illustrated preferred embodiments of the invention.

### BRIEF DESCRIPTION OF THE DRAWINGS

The invention may be more completely understood in consideration of the following detailed description of various embodiments of the invention in connection with the accompanying drawings, in which:

FIG. 1 illustrates one embodiment of the cartridge and the one way valve;

FIG. 2 illustrates a sectional plan view of an embodiment of the overall system;

FIG. 3 illustrates a sectional plan view of an alternative embodiment of the overall system;

FIG. 4 illustrates the charging system used for the current system;

FIG. 5 illustrates one embodiment of the spray apparatus with a propellant; and

FIG. 6 illustrates one embodiment for nasal usage.

### DETAILED DESCRIPTION OF THE INVENTION

The detailed description set forth below is intended as a description of presently preferred embodiments of the invention and does not represent the only forms in which the present invention may be construed and/or utilized. The description sets forth the functions and the sequence of the steps for producing the system and accompanying apparatus. However, it is to be understood that the same or equivalent functions and sequences may be accomplished by different embodiments also intended to be encompassed within the scope of the invention.

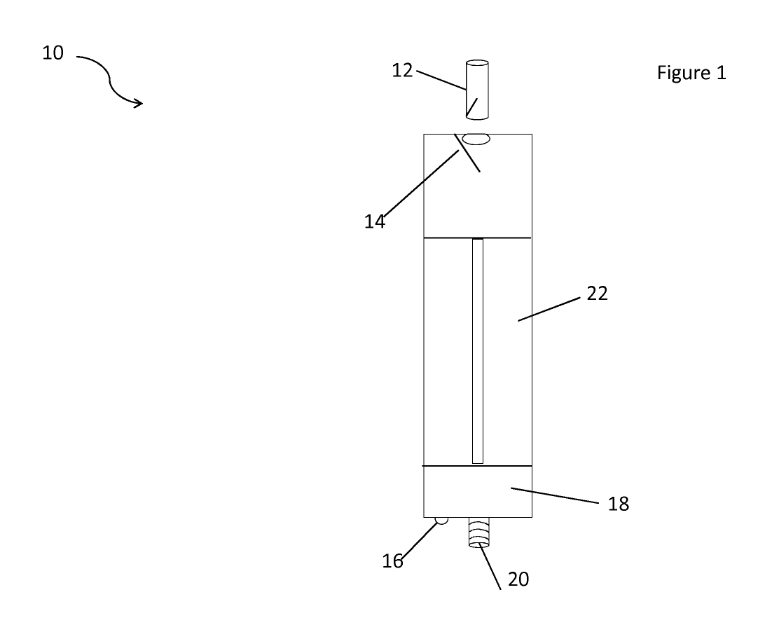

FIG. 1 illustrates one embodiment of the cartridge and one-way valve, 10. Illustrated is fully-sealed cartridge, 22, with a spring loaded valve, 14, a fill valve, 16, and an atomizer 18 located at the base of the cartridge with a threaded fastening mechanism 20. Also illustrated is a drip-tip 12 with a one-way valve.

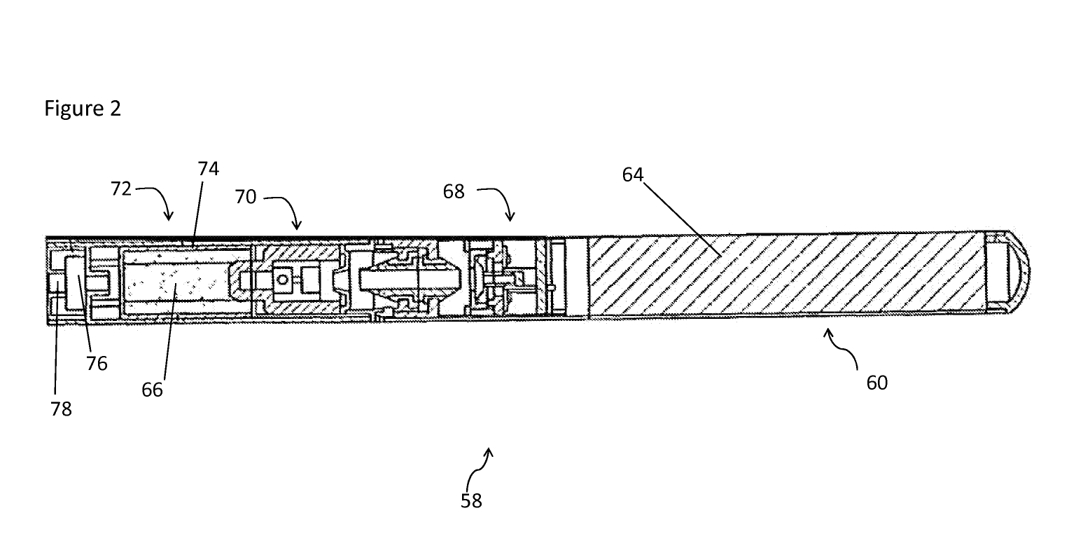

FIG. 2 is sectional plan view of an embodiment of the current system. The system includes an overall housing 58. The housing further includes an anterior end 72 and a distal end 60. The anterior end 72 of the housing is proximate to a user's mouth. The distal end 60 of the housing 58 is proximate to the user's mouth. The cartridge 74 includes an absorbent sponge 66, atomization means 70 and a liquid supplying bottle. The anterior end 72 of the housing includes a mouthpiece 78 and a filter 76. When the user inhales, the mouthpiece 78 is under negative pressure and the air pressure thus generated, released an actuating signal to an IC switch 68 to activate the power source. The power source further includes a smart chip (not shown), control panel (not shown) and a lithium ion rechargeable battery 64. The activated power source transfers heat to the liquid in the liquid supplying bottle. Vapor is generated when the heated liquid makes contact with the atomization means 70.

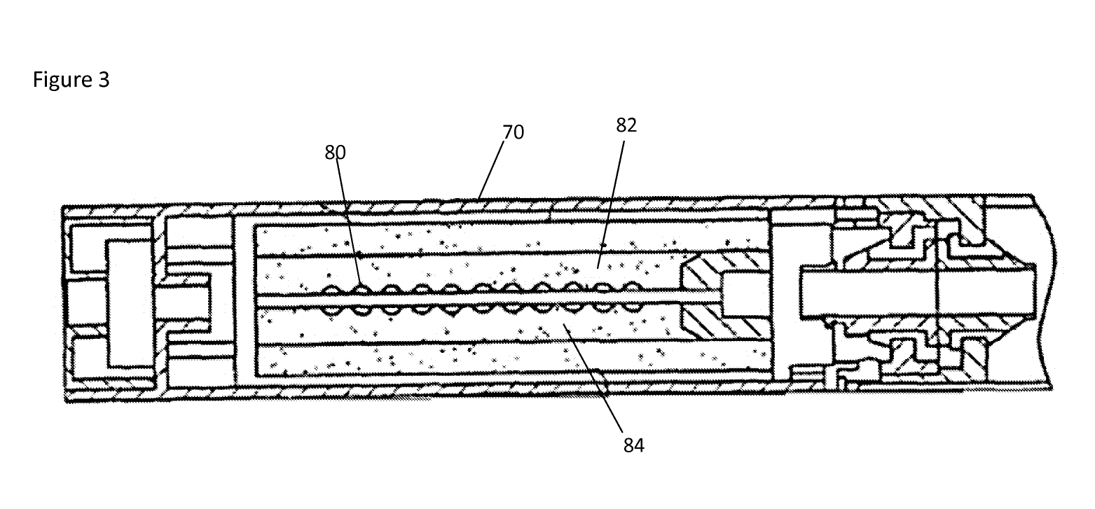

FIG. 3 illustrates an alternative embodiment in which a separation means, 80 divide the atomization means 70 in two upper and lower sub chambers, 82, 84 for generating additional vapor.

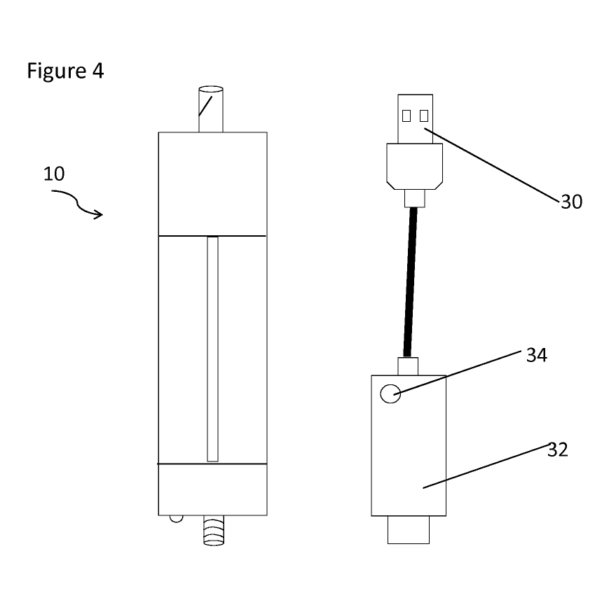

FIG. 4 illustrates the battery charging system. To charge the battery, remove the cartridge, 22 containing the liquid. Once the cartridge has been properly removed, gently screw the cartridge into the charger 32. With the battery attached to the charging cable, plug the cable which contains a USB end, 30 into any USB slot or use the wall plug and connect it to a 110 Volt electric outlet. There is an indicator light on the charging cable that will glow red while charging. Once the battery is done charging, the indicator light will glow green.

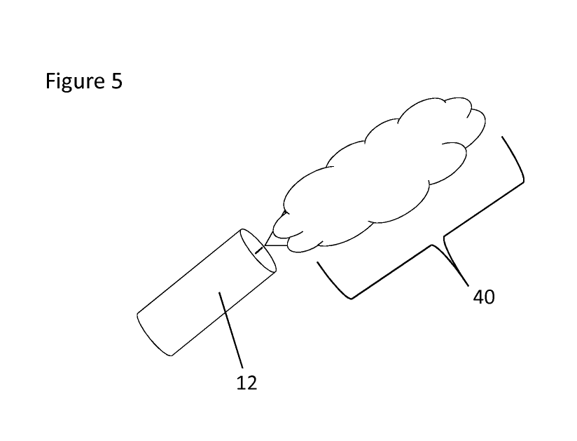

FIG. 5 illustrates the drip-tip 12 with a one-way valve dispensing a mist 40 that would be inhaled by the user.

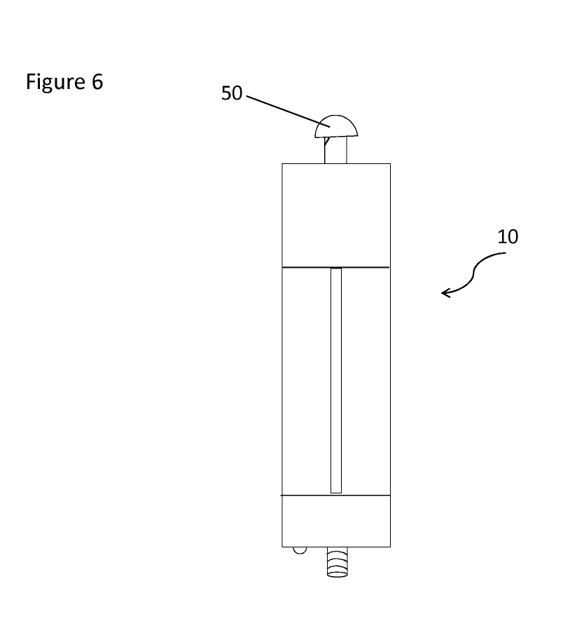

FIG. 6 illustrates a similar cartridge and one way valve but also includes a nasal usage embodiment 50 that is added to the drip-tip for nasal use.

In one embodiment, a cylindrical mechanism or an external tube made preferably the size, color and form of a cigarette, such tube having an outer end portion and an inner end portion, subdividing the interior space of the external tube is an internal shoulder which is preferably closer to the outer end portion than to the opposite inner end portion, thus dividing the tube into relatively short and substantially longer chambers.

The tube is supplied at the inner end portion with internal threads or some other means of connection for a mouthpiece as later described. In the outer end portion is detachably fitted a flavor cartridge of some suitable absorbent material, preferably having longitudinal spaced passages there through of a small diameter. The cartridge is abutted against the outer edge of the internal shoulder and is of an external diameter to fit snugly into the outer end portion so that it will be held therein by friction or other suitable means.

Within the more forward chamber of the outer tube is received a tubular liner preferably of insulating material and having an internal wall of a form and character to tumble the air or create turbulence therein. This internal wall may be spiraled or rifled as indicated. A mouthpiece is affixed to the inner end portion of the tube in any appropriate manner, preferably detachably as by external threads on the hollow shank of the mouthpiece which mate with the internal threads in the inner end portion of the outer tube.

The free outer end of the hollow shank, when fitted home in the outer tube, will encounter and push the insert against the inner wall of the internal shoulder 18 thus holding the insert immovably in place.

The hollow shank will preferably have an outstanding shoulder forwardly of the threads to engage the inner end of the tube in the completely assembled position of the mouthpiece relatively to the outer tube.

A cross-section through the hollow shank portion of the mouthpiece, a spider formation is shown providing air draft spaces between the hollow shank and an inner ring spaced inwardly from the hollow shank and connected therewith by radial arms. The air draft spaces communicate with the suction orifice of the mouthpiece at the inner end and at the outer end with the space circumscribed by the inner wall of the tubular insert.

Within the inner ring is a threaded electric socket and forwardly thereof a battery cavity for detachably receiving a battery having an inner contact and an outer contact with a contact strip between the inner contact and the socket.

The heating element is preferably a vacuum tube or bulb having a screw plug for detachable engagement with the socket.

The screw plug has an end contact adapted to close against the outer battery contact. The bulb or tube, similar to a light bulb, is preferably elongated and of a diameter to fit within the insert in such manner heating passage throughout the length of the bulb and around the complete circumference of the bulb in assembly, the flavor cartridge can be introduced and removed without regard to the other units of the device.

Before the mouthpiece is assembled to the external tube, the liner is slid through the open inner end portion of the tube until the outer end of the liner encounters the internal shoulder. The bulb will be mounted to the socket while the mouthpiece is detached from the tube whereupon the bulb may be introduced into the insert as the mouthpiece is put into place and rotated to effect attachment of the mouthpiece to the outer tube. The final home position of the parts is indicated in which the hollow shank engages the inner end of the insert while the outer end of the insert is abutted against the internal shoulder.

When the bulb is assembled to the socket its tip end will engage the inner battery contact and complete the circuit through the filament of the bulb by the contact strip. Thus the bulb will be illuminated or energized before assembly to the external tube.

The battery is removable so that when exhausted, a fresh dry battery may be substituted, access to the same being had by first unscrewing the mouthpiece and withdrawing the bulb and subsequently removing the bulb from its socket.

The insert is preferably of a ceramic material. By the act of inhalation through the mouthpiece, air from the ambient atmosphere is drawn in through the passages of the cartridge 20 which has been impregnated with suitable flavoring material which is picked up by the inhaled air and such air then passes into the heating chamber and is caused to flow around the heating bulb, the heated and flavored air finally passing through the mouth piece into the mouth and also, if desired, into the lungs.

An air circulation or recirculation system may be incorporated in order to cool the outer surface of the mechanism. The cartridge may contain a drip tip and a one-way valve to prevent the spread of disease if reused. The cartridge may be reusable or disposable, as may well be the tip mechanism.

The impregnation will be by a harmless flavored chemical compound. As suggested, such compounds may be solutions ranging from slightly mentholated water to a solution which would simulate artificially the flavor of menthol, or other lung soothing or cleansing substance or mixture.

A list of some herbals and nutraceuticals without regulatory requirements that may be administered through the instant system include, but are not limited to the following:

1) Lung Cleansing Kit (Menthol including the active ingredients of VicsVaporub)
2) Weight Control (Active ingredients of Hydroxicut) Sleep Aid (Melatonin, diphenhydramine and other OTC drugs)
3) Anxiety, depression, relaxant (Valerian Root Extract with or without Bay Leaf Extract)
4) Energy (Formulations used in HealthCare, sold in Whole food stores)
5) CoQ10 (High priced formulations sold in GNC etc.)
6) Pain (Naproxen, Ibuprofen other NSAIDs)
7) Sleep Aid (melatonin, diphenhydramine and other OTCs)
8) Migraine (will look up a Nutraceutical or Herbal)
For Prescription Drugs specifically
1) Migraine (sumatriptan)

### 2) Viagra

The system may further comprise variations such as the use of Lavender for the purposes of:
a. preventing panic attack and anxiety;
b. calming effect aids relaxation;
c. reducing stress; and,
d. relieving pain from tension headache.

In conclusion, herein is presented a lung cleaning system. The invention is illustrated by example in the flow diagrams and figures, and throughout the written description. It should be understood that numerous variations are possible, while adhering to the inventive concept. Such variations are contemplated as being a part of the present invention.

What is claimed is:

1. A vaporized lung cleansing system comprising:
a main elongated cylindrical body for enclosing said vaporized lung cleansing system, said body having an anterior and distal end;
a cartridge, having an atomization means, and a liquid supplying bottle wherein liquid in said liquid supplying bottle is in contact with said atomization means;
a power source having a smart chip, control panel and a lithium ion rechargeable battery for transferring heat to said liquid in said liquid supplying bottle;
a heating means within the distal end of said body,
a tubular liner in the body at least partially surrounding and spaced from the heating means and having:
an internal wall shaped to produce turbulence in an air stream drawn through a space between the heating means and tubular liner,
a mouthpiece on the anterior end of the body for drawing atmosphere air by inhaling through the anterior end of the body including the cartridge and through the space between the heating means and liner and into the mouth of the user, and said cartridge having air passages there through communicating at the outer end of the tube with the atmosphere and at the inner end of the tube with said space and ultimately with the mouthpiece for permitting air to be drawn past the flavor cartridge for inhalation by the user at the mouthpiece; and
an IC switch for releasing a signal to activate and deactivate said power source.

2. The vaporized lung cleansing system according to claim 1, wherein the elongated cylindrical body is comprised of a dense polymeric material.

3. The vaporized lung cleansing system according to claim 1, wherein said atomization means include an upper and lower sub chamber.

4. The vaporized lung cleansing system of claim 1 in which the internal wall of the tubular liner is rifled to produce the turbulent effect recited.

5. The vaporized lung cleansing system of claim 1 in which the tubular liner is of heat insulating material and in which the internal wall is spirally grooved to tumble the air around the heating means.

6. The vaporized lung cleansing system of claim 1 in which the mouthpiece further comprises a hollow shank detachably interlocked with the external tube and abutted against the inner end portion of the liner.

7. The vaporized lung cleansing system of claim 1 wherein the system delivers plain menthol to cleanse lungs.

8. The vaporized cleaning lung system of claim 1, wherein the mouthpiece has a drip tip comprises a one way valve.

9. The vaporized cleansing lung system of claim 8 wherein the one way valve comprises a spring loaded body.

10. The vaporized cleansing lung system of claim 1 wherein the cartridge is removable.

11. The vaporized cleansing lung system of claim 1 wherein the cartridge mechanism is fully sealed.

12. The vaporized cleansing lung system of claim 1 wherein the cartridge comprises a fill valve.

13. The vaporized cleansing lung system of claim 1 wherein the cartridge comprises a threaded fastening mechanism.

14. The vaporized cleansing lung system of claim 13 wherein the threaded fastening mechanism comprises 510 threading.

15. A method of infusing a vaporized lung cleansing system with medicinal substances comprising the steps of:
providing a vaporized lung cleansing system having a main elongated cylindrical body for enclosing said vaporized lung cleansing system and having an anterior and distal end; a cartridge, having atomization means and a liquid supplying bottle in contact with said atomization means; a power source having a smart chip, control panel, and lithium ion rechargeable battery for transferring heat to said liquid in the liquid supplying bottle; an IC switch for releasing a signal to activate and deactivate the power source; and
injecting a solution of medicinal substances into said liquid supplying bottle.

16. The method of infusing a vaporized lung cleansing system with medicinal substances of claim 15, wherein the medicinal substances are selected from the group consisting of lavender oil, caffeine, menthol, eucalyptus, glycerol beads, water, alcohol, nutraceuticals, herbals, prescription and non-prescription drugs, vitamins, vaccines, anti-pyretics, narcotic and non-narcotic pain relievers, diabetes medications, cystic fibrosis medications, obesity medications and anti-ageing medications.
